(19) United States
(12) Patent Application Publication
(10) Pub. No.: US 2009/0305394 A1
(43) Pub. Date: Dec. 10, 2009

(54) DNA CHIP FOR DIAGNOSIS OF CORNEAL DYSTROPHY

(75) Inventors: Sang Yup Lee, Daejeon (KR); So Young Yoo, Seoul (KR); Eung Kweon Kim, Seoul (KR); Ki-Chang Keum, Seoul (KR); Won-Min Yoo, Seoul (KR); Nae-Choon Yoo, Seoul (KR)

Correspondence Address: MOORE & VAN ALLEN PLLC, P.O. BOX 13706, Research Triangle Park, NC, 27709, US

(73) Assignee: MEDIGENES CO., LTD., Seoul (KR)

(21) Appl. No.: 12/160,965

(22) PCT Filed: Jan. 18, 2007
(86) PCT No.: PCT/KR07/00299

§ 371 Date: Nov. 14, 2008

(30) Foreign Application Priority Data
Jan. 18, 2006 (KR) 10-2006-0005550
Jan. 18, 2006 (KR) 10-2006-0005562

(51) Int. Cl. C07H 21/04; C12M 1/34

(52) U.S. Cl. 435/287.2; 536/23.1; 536/24.33

(57) ABSTRACT

The present invention relates to oligonucleotides for diagnosis of corneal dystrophy. More particularly, the present invention relates to oligonucleotides for detecting mutation of BIGH3 gene for diagnosis or corneal dystrophy including Avellino corneal dystrophy, which must be precisely diagnosed before vision correction surgery, and a DNA chip for diagnosis of corneal dystrophy, which has the oligonucleotides fixed thereon. According to the present invention, conventional microscopic diagnosis of corneal dystrophy can be replaced with a precise genetic method, which prevents a patient with corneal dystrophy from losing eyesight by eyesight correction surgery after erroneous diagnosis.

### TECHNICAL FIELD

The present invention relates to oligonucleotides for diagnosis of corneal dystrophy. More particularly, the present invention relates to oligonucleotides for detecting mutation of BIGH3 gene for diagnosis of corneal dystrophy including Avellino corneal dystrophy, which must be precisely diagnosed before vision correction surgery, and a DNA chip for diagnosis of corneal dystrophy, which has the oligonucleotides fixed thereon.

### BACKGROUND ART

Corneal dystrophy is a hereditary, autosomal dominant disease, which begins with a blurry symptom in the center of cornea and gradually spreads and thus ends up vision loss as a patient gets older. It includes Avellino corneal dystrophy, Granular corneal dystrophy, Lattice type I corneal dystrophy, Reis-bucklers corneal dystrophy, etc, and is caused by mutation of a gene coding βIG-H3.

Heterozygous patients suffering from Avellino corneal dystrophy appear to have severe loss of vision as getting older and homozygous patients appear to have complete loss of vision since 6 years old. Avellino corneal dystrophy is a newly named disease in 1988, divided from generally called Granular corneal dystrophy because it was found to have discrete symptoms and genetic foundation. And it has known to be the most common corneal dystrophy worldwide, 1/34011/1000 of prevalence rate in Korea (the case of heterozygote) based on genetic analysis indicates that it is a common dystrophy (Holland, E. J. et al., Opthalmology, 99:1564, 1992; Kennedy, S. M. et al., Br J. Opthalmol., 80:489, 1996; Dolmetsch, A. M. et al., Can. J. Opthalmol., 31:29, 1996; Afshari, N. A. et al., Arch. Opthalmol., 119:16, 2001; Stewart, H. S. Hum. Mutat., 14:126, 1999)

The present inventors has found that if a patient suffering from heterozygous Avellino corneal dystrophy has LASIK surgery, 2 years later, opacity of cornea starts to develop aggressively (Jun, R. M. et al., Opthalmology, 111:463, 2004) and eventually results in vision loss. Previously, eye surgery has been performed with an expectation that LASIK or Excimer Laser surgery would get rid of vision blurriness of a patient suffering from corneal dystrophy. And, even in Korea, approximately 3 hundred thousand cases of LASIK surgery have been performed, which leads to the assumption that 300 people lost their vision, based on 1/1000 of minimum estimation of heterozygous patients suffering from Avellino corneal dystrophy. Patients who have undergone LASIK surgery are mainly in their 20's and 30's carrying out productive activities; therefore, their vision loss causes serious troubles in both society and economics.

In addition, after approval of LASIK surgery in year 2000 in USA, African American patients suffering from Avellino corneal dystrophy who underwent LASIK surgery have been found to lose eye sight, which infers that plenty of similar cases might be occurring throughout the world.

Therefore, although accurate diagnosis of Avellino corneal dystrophy is required to prevent the progression of Avellino corneal dystrophy by LASIK surgery, the diagnosis of Avellino corneal dystrophy is just conducted by microscopic observation of corneal opacity and thus often doctors miss latent symptoms of patients to perform LASIK surgery, which results in vision loss. Therefore, rapid and precise diagnosis of corneal dystrophy is desperately in need.

Accordingly, the present inventors have made extensive efforts to develop a DNA chip for swiftly and accurately detecting mutations of BIGH3 gene that causes corneal dystrophy, and as a result, generated a probe comprising a specific mutation region of BIGH3 gene and manufactured a DNA chip on which the probe is fixed, and confirmed that corneal dystrophy can be effectively diagnosed using the DNA chip, thereby completing the present invention.

### SUMMARY OF INVENTION

The main object of the present invention is to provide oligonucleotides comprising a specific mutation region of BIGH3 gene that causes corneal dystrophy.

Another object of the present invention is to provide a DNA chip for diagnosis of corneal dystrophy, on which said oligonucleotides are fixed.

To achieve the above object, the present invention provides oligonucleotides for diagnosis of corneal dystrophy, which essentially comprises one or more nucleotide sequence selected from the group consisting of SEQ ID NO: 17, SEQ ID NO: 19, SEQ ID NO: 21, SEQ ID NO: 23, SEQ ID NO: 25, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 40, SEQ ID NO: 42, SEQ ID NO:44, SEQ ID NO:46, SEQ ID NO: 50, SEQ ID NO:53 and SEQ ID NO: 59.

In the present invention, the oligonucleotide essentially comprising the nucleotide sequence of SEQ ID NO: 50 is preferably set forth in SEQ ID NO: 12, and the oligonucleotide essentially comprising the nucleotide sequence of SEQ ID NO: 53 is preferably set forth in SEQ ID NO: 13.

In the present invention, the oligonucleotide essentially comprising the nucleotide sequence of SEQ ID NO: 59 is preferably set forth in SEQ ID NO: 15, and the oligonucleotide essentially comprising the nucleotide sequence of SEQ ID NO: 35 is preferably set forth in SEQ ID NO: 65.

In the present invention, the length of the oligonucleotides is 13 to 17 bp.

The present invention also provides a DNA chip for diagnosis of corneal dystrophy, on which the oligonucleotides are fixed.

In the present invention, the DNA chip for diagnosis of corneal dystrophy preferably additionally immobilizes oligonucleotides essentially comprising one or more nucleotide sequences selected from the group consisting of SEQ ID NO: 16, SEQ ID NO: 18, SEQ ID NO: 20, SEQ ID NO: 22, SEQ ID NO: 24, SEQ ID NO: 26, SEQ ID NO: 32, SEQ ID NO: 34, SEQ ID NO: 36, SEQ ID NO: 38, SEQ ID NO: 39, SEQ ID NO: 41, SEQ ID NO: 43, SEQ ID NO: 45, SEQ ID NO: 47, and SEQ ID NO:56, thereon.

In the present invention, the oligonucleotide essentially comprising the nucleotide sequence of SEQ ID NO: 47 is preferably set forth in SEQ ID NO: 11, the oligonucleotide essentially comprising the nucleotide sequence of SEQ ID NO: 56 is preferably set forth in SEQ ID NO: 14, and the oligonucleotide essentially comprising the nucleotide sequence of SEQ ID NO: 34 is preferably set forth in SEQ ID NO: 62.

In the present invention, the DNA chip for diagnosis of corneal dystrophy preferably has all oligonucleotides, essentially comprising the nucleotide sequence of the group consisting of SEQ ID NO: 16 to SEQ ID NO:47, SEQ ID NO: 50, SEQ ID NO: 53, SEQ ID NO: 56 and SEQ ID NO: 59, fixed thereon.

The present invention also provides a DNA chip for diagnosis of corneal dystrophy, which has all oligonucleotides of the group consisting of SEQ ID NO: 11 to SEQ ID NO: 15, SEQ ID NO: 62 and SEQ ID NO: 65 fixed thereon.

The present invention also provides a pair of primers selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; and SEQ ID NO: 9 and SEQ ID NO: 10.

Other features and embodiments of the present invention will be more fully apparent from the following detailed description and appended claims.

### BRIEF DESCRIPTION OF DRAWINGS

FIG. 1 shows spots on a DNA chip to measure detection efficiency of DNA chip depending on spotting solutions.

FIG. 2 shows hybridization results after applying exon 4 PCR product from normal individuals to the DNA chip of FIG. 1.

FIG. 3 shows hybridization results after applying exon 4 PCR product from patients with heterozygous Avellino corneal dystrophy to the DNA chip of FIG. 1.

FIG. 4 shows hybridization results after applying exon 4 PCR product from patients with homozygous Avellino corneal dystrophy to the DNA chip of FIG. 1.

FIG. 5 shows hybridization results after applying exon 4 PCR product from patients with heterozygous Reis-bucklers corneal dystrophy (CD1) to the DNA chip of FIG. 1.

FIG. 6 illustrates spot arrangement of a DNA chip designed depending on the concentration of probes.

FIG. 7 shows the anticipated result of hybridization according to each patient when the DNA chip of FIG. 6 was used.

FIG. 8 shows hybridization results depending on hybridization time of each primer set when the DNA chip of FIG. 6 was used.

FIG. 9 displays graphs of hybridization results of the DNA chip depending on the length of probes.

FIG. 10 illustrates spot arrangement of the DNA chip to estimate hybridization efficiency of DNA chip depending on probe length.

FIG. 11 shows hybridization results of the DNA chip depending on the length of probes.

FIG. 12 illustrates spot arrangement of the DNA chip manufactured with 50 μM of 15 mer probe using 3×SSC spotting solution.

FIG. 13 shows hybridization results of the DNA chip of FIG. 12.

### DETAILED DESCRIPTION OF THE INVENTION AND PREFERRED EMBODIMENTS

The present inventors have paid attention to the fact that BIGH3 gene mutation causes corneal dystrophy having the symptom of corneal opacity and constructed a probe for diagnosing each corneal dystrophy, based on point mutations in BIGH3 gene shown in patients of various corneal dystrophies such as Avellino corneal dystrophy, Lattice corneal dystrophy, Granular corneal dystrophy, Reis-bucklers corneal dystrophy.

The probes constructed in the present invention comprise nucleotide sequences of SEQ ID NO: 11 to SEQ ID NO: 67.

In order to manufacture a DNA chip capable of effectively detecting corneal dystrophy using the probe, optimal spotting solution, optimal time for immobilization, and the optimal length of probes were examined. Consequently, best result was generated by using 3×SSC as a spotting solution for 6 hours of hybridization time with 15 mer probe for detecting exon 4 mutation or with 17 mer probe for detecting exon 12 mutation in patients with corneal dystrophy, respectively.

The following definitions serve to illustrate the terms and expressions used in the different embodiments of the present invention as set out below.

An “isolated” nucleic acid molecule is one separated from other nucleic acid molecules that are present in the natural source of the nucleic acid. For example, with regards to genomic DNA, the term “isolated” includes nucleic acid molecules separated from the chromosome with which the genomic DNA is naturally associated.

The term “probe” or “nucleic acid probe” refers to single-stranded and sequence specific oligonucleotides, the sequence of which is sufficiently complementary to hybridize to a target nucleotide sequence to be detected

By “composition” is meant that a probe complementary to prokaryotic or eukaryotic genetic sequences may be a purified nucleic acid or in combination with other probes. In addition, the probe may be in a dry state in combination with salt or buffer, and may be a precipitate in an alcohol solution or in an aqueous solution.

The term “target” refers to nucleic acid molecules derived from a biological sample, which has a nucleotide sequence complementary to any one of the nucleic acid probes of the present invention. The target nucleic acid can be single- or double-stranded DNA (optionally obtained after amplification) or RNA, and contain a sequence at least partially complementary to at least one oligonucleotide probe.

The phrase “a biological sample” refers to a specimen such as clinical samples (e.g., pus, sputum, blood, urine, etc.), environmental samples, bacterial colonies, contaminated or pure culture products, purified nucleic acid, etc., in which the target sequence of interest is confined.

By “oligonucleotide” is meant a nucleotide polymer generally about 10 to about 100 nucleotides in length, but the length may be longer than 100 or shorter than 10 nucleotides.

By “nucleotide” is meant a subunit of a nucleic acid consisting of a phosphate group, a 5-carbon sugar and a base containing nitrogen. In RNA, the 5-carbon sugar is ribose. In DNA, it is 2-deoxyribose. For a 5-nucleotide, the sugar contains a hydroxyl group (—OH) at the second carbon. The term also includes analogs of such subunits the same as a methoxy group at the second carbon of ribose.

The term “homologous” is synonymous with identical, meaning that polynucleic acids e.g., 90% homologous have 90% identical base pairs in the same position, when sequences are aligned.

“Hybridization” involves annealing of a complementary sequence to its target nucleic acid (a sequence to be detected). The ability of two polymers of nucleic acids containing complementary sequences to recognize each other and anneal through base pairing interaction is a well-known phenomenon.

The term “primer” refers to a single-stranded oligonucleotide sequence of DNA providing an initiation point for synthesis and elongation of a product that is complementary to a strand of nucleic acid to be copied. The length and the sequence of a primer are recommended as much as they allow priming the synthesis and elongation of products. Preferably, the primer is about 5-50 nucleotides long. Specific length and sequence will depend on the complexity of target DNAs or RNAs, as well as on ambient conditions for primer use such as temperature and ionic strength.

The term “label” as used herein refers to any atom or molecule that can be used to produce a detectable (preferably quantifiable) signal and attached to a nucleic acid.

Labels may provide detectable signals of fluorescence, radioactivity, colorimetry, gravimetry, X-ray diffraction or absorption, magnetism, and the like.

By “hybrid” is meant the complex formed between two single stranded nucleic acid sequences by Watson-Crick base pairing or non-canonical base pairing between the complementary bases.

The phrase “probe specificity” refers to characteristic of a probe, describing its ability to distinguish between a target and a non-target sequence. In this regard, the term “specific” means that a nucleotide sequence will hybridize to a defined target sequence and will substantially not or minimally hybridize to a non-target sequence. Probe specificity is dependent on sequence and assay conditions.

The phrase “a standard strain” includes a microorganism commercially or readily available in the art.

### Identification of Probes

To produce nucleic acid probes that can detect various prokaryotic or eukaryotic genetic sequences, each detection probe needs to be specific to a target sequence. For this, screening of probe candidates specific to each target gene or organism is preceded.

Probe candidates are selected in the region of a gene embracing their target sequences. Specificity of probe candidates is first examined by BLAST search, comparing homology of each nucleotide sequence, confirmed by hybridization using the “target” in vitro. And probes that only anneal to target genes among candidates are finally determined for gene identification.

Additionally, sensitivity of the probes for gene identification, selected by the procedure above, is examined by clinical trials using a variety of biological samples.

The probe of the present invention are 11˜17 mer oligonucleotides, and preferably 70%, 80%, 90% or more than 95% homologous and exactly complementary to their target sequences to be detected. The length of the inventive probe for detecting and identifying each “target” may be or more than 50 nucleotides. The nucleotides used in the present invention may include ribonucleotides, deoxyribonucleotides and modified nucleotides such as inosine or nucleotides containing analogue groups, but should not alter their hybridization characteristics.

### Use of Probes

The probes of the present invention can be used for all the well-recognized hybridization techniques investigating presence or absence of a target nucleic acid in a biological sample for diagnostic purposes, such as a point-spotting technique on a filter called “Dot-blot” (Maniatis et al., Molecular Cloning, Cold Spring Harbor, 1982), Southern Blot (Southern, E. M., J. Mol. Biol. 98, 503 (1975)), or Northern Blot.

The probes of the invention can also be used in sandwich hybridization system that enhances specificity of a nucleic acid probe-based assay. The principle and the procedure of sandwich hybridization in a nucleic acid probe-based assay have been already described (Dunn and Hassel, Cell, 12: 23-36; 1977; Ranki et al., Gene, 21: 77-85; 1983). The sandwich hybridization technique uses a capture probe and/or a detection probe. Those probes may hybridize with two different regions of their target nucleic acid, and at least one of the probes (generally the detection probe) may hybridize with a target region specific to a target species. It is understood that the capture probe and the detection probe must have at least partly distinct nucleotide sequences.

Although a direct hybridization assay has favorable kinetics, sandwich hybridization is advantageous with respect to a high signal-to-noise ratio. Moreover, sandwich hybridization can improve specificity of a nucleic acid probe-based assay. The incubation and subsequent washing steps that constitute the key steps of the sandwich hybridization process are each carried out at a constant temperature between about 20° C. and 65° C. It is known that nucleic acid hybrids have a dissociation temperature which depends on the number of hybridized bases (the temperature tends to rise in proportion to the size of the hybrid), and which also depends on the nature of hybridized bases and their adjacent bases. The hybridization temperature used in sandwich hybridization technique should be chosen below the half-dissociation temperature relating to a given probe and its complementary target sequence. And the temperature may be determined by simple routine experiments.

The probes of the present invention can also be used in a competition hybridization protocol. In competition hybridization, a target molecule competes for hybrid formation with a specific probe and its complement. The more targets exist, the less amount of probe-complement hybrids becomes. A positive signal indicating existence of a specific target tends to decline in hybridization reaction as compared with a system without a target. In a particular embodiment, a specific oligonucleotide probe, conveniently labeled, is hybridized with its target molecule. Next, the mixture is transferred to a microtiter dish well, in which complementary oligonucleotides and the specific probe are fixed, and allowed to keep all hybridized. After washing, the hybrids of the complementary oligonucleotides and the probes are measured preferably quantitatively based on used label.

In addition, the probes of the present invention can be used in reversed hybridization (Proc. Natl. Acad. Sci. USA, 86: 6230-6234, 1989). For the method, a target sequence can first be enzymatically amplified by PCR using 5′-biotinylated primers. In a second step, the amplified products are detected upon hybridization with specific oligonucleotides immobilized on a solid support. Reversed hybridization may also be carried out without amplification steps. In that particular case, prior to hybridization, the nucleic acids present in a biological sample have to be labeled or modified, specifically or non-specifically by addition of certain dyes or chemical reaction.

The nucleic acid probes of the present invention may be included in a kit that can apply to rapidly determine presence or absence of pathogenic species of interest. The kit includes all components necessary for assay determining presence of a target gene. In the universal concept, the kit includes a stable reagent containing labeled probes, a hybridization solution in either dry or liquid form for hybridizing target and probe polynucleotide, a solution for washing and removing undesirable and unbound polynucleotide, a substrate for detecting labeled duplex, and optionally an instrument for detecting the label.

One specific embodiment of the present invention embraces a kit that utilizes the concept of sandwich assay. This kit includes a first component for collecting samples from patients including a scraping device or paper points, vials for containment, and buffers for dispersing and dissolving the sample. A second component includes a medium in either dry or liquid form for hybridizing target and probe polynucleotide and a medium for washing out to remove undesirable and unbound polynucleotide. A third component includes a solid support, on which or to which nucleic acid probe(s) is conjugated, which is unlabeled and complementary to part of target polynucleotide. In the case of multiple target analysis, more than one capture probe, each specific to its own ribosomal RNA, is applied to different discrete regions of dipstick.

A fourth component contains a labeled probe that is complementary to different regions from the same rRNA strand hybridizing to the immobilized and unlabeled nucleic acid probe of the third component. The probe components described herein include combination of probes in dry form such as lyophilized nucleic acid or in precipitated form such as alcohol-precipitated nucleic acid, or in a buffered solution. The label may be any of the labels described above.

For example, probes can be biotinylated using conventional ways, and presence of a biotinylated probe can be detected by adding an avidin-conjugated enzyme such as horseradish peroxidase, and then supplying a substrate of peroxidase that can be monitored visually or by a colorimeter or a spectrophotometer. This labeling method and other enzyme-conjugating labels have advantages of being economical, highly sensitive, and relatively safe, compared with radioactive labeling methods. Kits of assorted components include various reagents for detection by labeled probes, an instruction, containers for mixing and reacting positive and negative controls, etc.

### DNA Chip

The probes of the present invention are also used in a DNA chip. In a preferred embodiment, the present invention provides a DNA chip that nucleic acid probes are immobilized on its solid support. DNA chip, a narrow support that is mounted with various fragments of nucleotides with high density, is applied to identify DNA information by hybridizing unknown DNA extracted from unknown samples with immobilized DNA thereof. Examples of solid supports for holding probe oligonucleotides include inorganic materials such as glass and silicon, and polymeric materials such as acryl, polyethylene terephthalate (PET), polystyrene, polycarbonate and polypropylene. The surface of solid substrates may be flat or highly porous. The probes are immobilized by covalent bond of either 3′-end or 5-′end on the substrate. The immobilization can be achieved by conventional techniques, for example, using electrostatic force, fixing amine group-attached oligomers on aldehyde-coated slide, or spotting on an amine coated slides, L-lysine-coated slides or nitrocellulose-coated slides. In one embodiment of the present invention, a base with an amine group was incorporated into 3′ position of the probe during its synthesis, which makes it easy to bind covalently onto aldehyde-coated glass slides.

Immobilization and arrangement of various probes onto solid substrates are carried out by pin microarray, inkjet, photolithography, electric array, etc. In an embodiment of the invention, probes are separately dissolved in a buffer solution, and the resulting solution is spotted onto the substrate by using a microarrayer prepared by a known method (Yoon et al., J. Microbiol. Biotechnol., 10(1), 21-26, 2000).

The principle of microarrayer is that minutely constructed pins pick probe DNA from a reservoir and transfer to one site on a support appointed by a computer. For fixing the probes transferred by a microarrayer, immobilization reaction proceeds at least for one hour under humid condition of from 45% to 65%, preferably, from 50% to 55%, and it was left to stand at least for 6 hours to induce reaction between an amine group at 3′ end of the probe and an aldehyde group coated onto the glass slide.

For detecting cells derived from living organisms or a living organism itself, the cells occasionally need partial and/or total lysis by a chemical and/or a mechanical procedure for easy access to their RNA and/or DNA, and then they are brought in contact with one or several probes of the present invention. The contact may be carried out on an appropriate support such as nitrocellulose, cellulose or a nylon filter in a solution, or in a liquid medium, which may take place under a suboptimal, an optimal or a restrictive condition. Such conditions include temperature, concentration of reactants, presence of substances lowering an optimal temperature of nucleic acid pairing (e.g., formamide, dimethylsulfoxide and urea), and presence of substances apparently lowering reaction volume and/or accelerating hybrid formation (e.g., dextran sulfate, polyethyleneglycol or phenol).

### Preparation of Probes

Traditional cloning methods may produce large quantities of nucleic acid probes by either gene manipulation of the desired sequence as described in Maniatis, T, et al. Molecular Cloning: A Laboratory Manual, Cold Spring Harbor, New York, 1982, or by chemical synthesis using a commercially available DNA synthesizer.

The probes of the present invention can be prepared to be single-stranded or double-stranded by conventional methods. A representative example of preparing a single-stranded probe is to synthesize probes comprising a desired number of nucleotides after dimethoxytrityl (DMT) off method by an automated DNA synthesizer and stripping protection.

One end of the probes, while synthesized, is labeled with a fluorescent dye (fluorescein isothiocyanate, FITC) to confirm presence or absence of nucleic acids of interest. Alternatively, A DNA probe complementary to a single-stranded DNA template is prepared by annealing primers to its template DNA and polymerizing with Klenow enzyme and fluorescent-labeled dNTP. The probes, thus exhibit high sensitivity and specificity owing to the fluorescent dye.

For a preparation of a double-stranded probe, genomic DNA or plasmid DNA is digested with specific restriction enzymes to obtain probes comprising desired regions of a gene or a nucleotide fragment. Random priming method is one way of polymerizing fluorescent-labeled probes with various lengths by six random hexamers. Alternatively, Probes can be synthesized by attaching ³²P to 5′-end of DNA with T4 polynucleotide kinase and also synthesized by generating nicks in double-stranded DNA molecules with DNase I, polymerizing DNA with DNA polymerase I and fluorescent-labeled dNTP. The synthetic double-stranded probes are denatured to make it single-stranded, which is then used for hybridization.

The probes of the present invention may be advantageously labeled. Any conventional label can be utilized. The probes can be labeled by means of radioactive tracers such as ³²P, ³⁵S, ¹²⁵I, ³H and ¹⁴C. The radioactive labeling can be carried out according to any conventional method such as terminal labeling at the 3′ or 5′ end using a radiolabeled nucleotide, polynucleotide kinase, terminal transferase, or ligase.

Another method for radioactive labeling is chemical iodination of probes of the present invention attaching several ¹²⁵I on the probes. If one of the probes of the present invention is radioactive, detection of ionizing ray is generally conducted by autoradiography, liquid scintillation, gamma counting or any other conventional method.

The probes of the present invention can be labeled by means of non-radioactive residues featuring: immunological properties (e.g., antigens or haptens); specific affinity for some reagents (e.g., ligands); detectable properties by enzymatic reaction (e.g., enzymes, co-enzymes, enzyme substrates or intermediates taking part in an enzymatic reaction); and physical properties including fluorescence, emission or absorption of light at a certain wavelength. Antibodies can be used to specifically detect a hybrid of probes and targets. Non-radioactive labels can be provided when the probe of the present invention is chemically synthesized. Adenosine, guanosine, cytidine, thymidine and uracyl residues are easily coupled with other chemical residues, enabling to detect the probe, or hybrids of probes and their complementary DNA or RNA fragments.

### Target

Nucleic acids are extracted from a sample to provide nucleic acid substrates for diagnosis of diseases with biological samples. The nucleic acids may be extracted from a variety of clinical samples using standard techniques or commercially available kits. For example, kits for isolating RNA or DNA from tissue samples are available from Qiagen, Inc. (Chatsworth, Calif., USA) and Stratagene (La Jolla, Calif., USA). For example, QIAamp Blood extraction kit can isolate DNA from blood, bone marrow, body fluids or cell suspensions. QIAamp tissue extraction kit can purify DNA from tissues such as muscles and organs.

If double-stranded, target DNA is recommended to denature prior to proceeding detection. Denaturation of double-stranded DNA can be conducted by chemical, mechanical or enzymatic method heating up to a proper temperature over 80° C.

Target DNA hybridizing to a probe is usually prepared by two kinds of methods. A first method is Southern blot or Northern blot in which genomic DNA or plasmid DNA is digested with appropriate restriction enzymes, and digested DNA fragments are separated and purified by using agarose gel electrophoresis. A second method is amplification of desired DNA regions by PCR.

Examples of PCR include: typical PCR using same amounts of forward and reverse primers; Asymmetric PCR using different amount of primers and resulting in double-stranded and single-stranded bands; Multiplex PCR using various primer pairs and amplifying different regions simultaneously; Ligase chain reaction (LCR) using specific 4 primers and ligase and measuring fluorescence intensity by ELIA (Enzyme Linked Immunosorbent Assay); besides, Hot Start PCR, Nest-PCR, DOP-PCR (Denaturated oligonucleotide primer PCR), RT-PCR (Reverse Transcription PCR), Semi-quantitative RT-PCR, Real time PCR, RACE (Rapid Amplification of cDNA Ends), Competitive PCR, STR (Short Tandem Repeats), SSCP (Single Strand Conformation Polymorphism), DDRT-PCR (Differential Display Reverse Transcriptase), etc.

In one preferred embodiment of the present invention, asymmetric PCR is performed to polymerize a gene fragment using DNA extracted from a specimen as a template. The gene fragment is polymerized by one reaction of PCR using different amount of forward and reverse primers in a ratio of 1:5.

In a preferred embodiment of the PCR, 5 ul of 10×PCR buffer solution (100 mM Tris-HCl, pH 8.3, 500 mM KCl, 15 mM MgCl₂), 4 ul of dNTP mixture (dATP, dGTP, dCTP, dTTP, each 2.5 mM), 0.5 ul of 10 pmole forward primer, 2.5 ul of 10 pmole reverse primer, 1 ul of 1/10 diluted template DNA (100 ng) and 0.5 ul of Taq polymerase (5 unit/ul, Takara Shuzo Co., Shiga, Japan) are mixed and distilled water is added to the cocktail to be up to 50 ul. The PCR is performed by following steps: First denaturation at 94° C. for 7 min once, 10 cycles of second denaturation at 94° C. for 1 min, annealing at 52° C. for 1 min and extension at 72° C. for 1 min; 30 cycles of third denaturation at 94° C. for 1 min, annealing at 52° C. for 1 min and extension at 72° C. for 1 min, and then 72° C. for 5 min only at the last extension. The PCR products are confirmed by agarose gel electrophoresis.

### Hybridization and Washing

A particular hybridization technique is not essential to the present invention. Hybridization techniques are generally described in Nucleic Acid Hybridization: A Practical Approach, Ed. Hames, B. D. and Higgins, S. J., IRL Press, 1987; Gall and Pardue (1969), Proc. Natl. Acad. Sci., U.S.A., 63: 378-383, and John, Burnsteil and Jones (1969) Nature, 223: 582-587.

The hybridization conditions are determined by the “stringency” that is to say the strictness of the operating conditions. The more stringent a condition is, the more specific probes and targets hybridize. Stringency indicates especially the base composition of a probe/target duplex as well as the degree of mismatch between nucleic acids. Stringency can likewise reflect functional parameters of hybridization reaction, such as concentration and type of ionic species present in a hybridization solution, nature and concentration of denaturing agents and/or hybridization temperature. Stringency of a hybridization condition depends especially on probes used. All data about the conditions are well known, and an appropriate condition of individual hybridization may be established by routine experiments. With regard to the length of probes, temperature of hybridization reaction generally lies in approximately within the range of about 20° C. and 65° C., in particular, between 35° C. and 65° C. in about 0.8 to 1M of a saline solution.

Nucleic acid hybridization of labeled oligonucleotide probes and nucleic acid targets can be enhanced by the use of “unlabeled Helper Probes” as disclosed in U.S. Pat. No. 5,030,557. Helper probes are oligonucleotides that bind to another region of nucleic acid, not where the assay probe is targeting. The helping interaction of additional probes accelerates the binding of assay probes, forming new secondary and tertiary structures on the targeted region of single stranded nucleic acids.

It will be appreciated by those skilled in the art that factors affecting thermal stability of probe/target hybrids can also affect probe specificity. Thus, the melting profile, including melting temperature (Tm) of probe/target hybrids needs to be determined. The preferred method is described in U.S. Pat. No. 5,283,174.

Tm of probe/target hybrids may be determined by a hybridization protection assay as follows. With the excess amount of targets, probe/target hybrids are formed in a lithium succinate buffered solution containing lithium lauryl sulfate. Each aliquot of the “preformed” hybrid is diluted in hybridization buffer and incubated for five minutes at various temperatures, gradually increasing 2-5° C. from below anticipated Tm (typically 55° C.). The solutions are then diluted with a mildly alkaline borate buffer and incubated at lower temperature (for example, 50° C.) for ten minutes. Under this condition, while the acridinium ester attached to single-stranded probes is hydrolyzed, the acridinum ester attached to hybridized probes is relatively protected. This is referred to as a hybridization protection assay (HPA). The amount of chemiluminescence's remnants is proportional to the amount of hybrids, and is measured in a luminometer after adding hydrogen peroxide and alkali in order. The data is plotted by percentage of maximum signal (usually from the lowest temperature) versus temperature.

Tm is defined by a point reaching at 50% of maximum signal. As an alternative, Tm of a probe/target hybrid may also be determined by isotopic methods well known to those skilled in the art. It should be noted that Tm of a given hybrid may vary depending on the concentration of salts, detergents and other solutes existing in hybridization solution, which affect relative stability of the hybrid during thermal denaturation (Molecular Cloning: A Laboratory Manual Sambrook et al., eds. Cold Spring Harbor Lab Publ., 9.51 (2nd ed., 1989)).

Hybridization conditions can be monitored by several parameters, e.g., hybridization temperature, nature and concentration of components in the media, and temperature at which hybrids are washed. Hybridization and washing temperature are limited by maximum temperature corresponding to probe's nucleic acid composition, composition and length, and the maximum temperature of probes for hybridization or wash described herein is about 30° C. to 60° C. At higher temperature, the duplexing competes with the dissociation or denaturation of probe/target hybrid. A preferred hybridization medium contains about 3×SSC (1×SSC=0.15 M NaCl, 0.015 M sodium citrate, pH 7.0), about 25 mM of phosphate buffer pH 7.1, and 20% deionized formamide, 0.02% Ficoll, 0.02% bovine serum albumin, 0.02% polyvinylpyrrolidone and about 0.1 mg/ml sheared denatured salmon sperm DNA. A preferred wash medium contains about 3×SSC, 25 mM phosphate buffer pH 7.1 and 20% deionized formamide. However, when modifications are introduced either in the probes or in the media, the temperatures, suitable for hybridization to bring out required specificity, should change based on known relationships as described in the following reference: B. D. HAMES and S. J. HIGGINS, (eds.). Nucleic acid hybridization. A practical approach, IRL Press, Oxford, U.K., 1985.

In this respect, it should also be noted that, generally because DNA:DNA hybrids are less stable than RNA:DNA or RNA:RNA hybrids, their conditions of hybridization need to be carefully adjusted to achieve specific detection depending on hybrid features.

In a preferred embodiment of the present invention, amplified target fragments of a gene is added in a hybridization buffer solution (6×SSPE (0.15M NaCl, 5 mM C₆H₅Na₃O₇, pH 7.0), 20% (v/v) formamide), applied onto a glass slide holding probes, and then incubated at 30° C. for 6 hours, so that said probes can complementarily hybridize with said targets. The glass slide is washed sequentially with 3×SSPE, 2×SSPE and 1×SSPE for 5 min.

Hybrids can be quantified by labeling targets with a fluorescence chemical or a radioactive isotope in accordance to conventional methods. Label incorporation may be carried out by using labeled primers or labeled nucleotides during polymerization and amplification.

### EXAMPLES

The present invention will hereinafter be described in further detail by examples. It will however be obvious to a person skilled in the art that these examples are given for illustrative purpose only, and the scope of the present invention is not limited to or by the examples.

All the following experiments in Examples were approved by IRB (Institutional Review Board) in Severance hospital in Korea and conducted by prior agreement of every attendee in accordance with the declaration of Helsinki.

### Example 1

### Determining Mutation Types of BIGH3 Protein

In order to construct diagnostic probes for detecting genetic mutation of BIGH3, one of the causes of ophthalmologic diseases including Avellino corneal dystrophy, its mutation sites were determined to produce probes.

The mutation sites of BIGH3 protein were identified, ensuring their amino acid and nucleotide sequences by GenBank and OMIM (Online Medelian Inheritance in Man), databases of nucleotide sequences of NCBI (National Center for Biotechnology Information), and allele information of each disease was comprehended. The types of mutation to be searched were determined to test efficacy of DNA chip. Especially among hot spots for BIGH3 gene mutation, mutation regions causing Avellino dystrophy, Lattice type I dystrophy and Reis-bucklers I were selected (Table 1).

TABLE 1
Ophthalmologic diseases caused by genetic mutation of BIGH3

| | Mutated | | |
|---|---|---|---|
| Phenotype | Amino acid | Sequence change | Exon |
| Avellino dystrophy | R124H | CGC → CAC | 4 |
| Granular dystrophy | R555W | CGG → TGG | 12 |
| Lattice dystrophy | R124C | CGC → TGC | 4 |
| Lattice dystrophy | L518P | CTG → CCG | 12 |
| Lattice dystrophy | L527R | CTG → CGG | 12 |
| Lattice dystrophy | H626R | CAT → CGT | 14 |
| Lattice dystrophy | N622H | AAC → CAC | 14 |
| Lattice dystrophy | N544S | AAT → AGT | 12 |
| Lattice type IIIA | P501T | CCA → ACA | 11 |
| Lattice type IIIA | A546T | GCC → ACC | 12 |
| Reis-bucklers I | R124L | CGC → CTC | 4 |
| (Reis-bucklers(CDB1)) | | | |
| Reis-bucklers | F540 deletion | TTT → — | 12 |
| Reis-bucklers (CDB2) | R555Q | CGG → CAG | 12 |
| Reis-bucklers | G623D | GGC → GAT | 14 |

### Example 2

### Obtaining Search Regions by PCR

In order to search all mutations shown in Table 1, five pairs of primers encompassing exon 4, exon 11, exon 12 and exon 14 were designed. Among them, two pairs of primers were used to amplify the mutation region of exon 4. One of the pairs (Primer 1 and Primer 2) was concluded suitable for experiments of diagnostic DNA chip, and the other was designed for direct DNA sequence analysis (Primer 3 and Primer 4). In addition, the DNA probes and their complementary primers (reverse primers) to be searched were labeled with a fluorescent chemical on their 5′-hydroxyl group. By Cy5 and Cy3 that were bound to primer 2 and primer 4, respectively, those primer pairs were hence effectively distinguished.

TABLE 2
Primers for amplifying genetic regions
encompassing mutation sites

| | SEQ | |
|---|---|---|
| Primer # | ID NO. | Sequence (5′ → 3′) |
| 1 | 1 | agc cct acc act ctc aa |
| 2 | 2 | cag gcc tcg ttg cta ggg |
| 3 | 3 | ccc cag agg cca tcc ctc ct |
| 4 | 4 | ccg ggc aga cgg agg tca tc |
| 5 | 5 | ctc gtg gga gta taa cca gt |
| 6 | 6 | tgg gca gaa gct cca ccc gg |
| 7 | 7 | cat tcc agt ggc ctg gac tct act atc |
| 8 | 8 | ggg gcc ctg agg gat cac tac tt |
| 9 | 9 | ctg ttc agt aaa cac ttg ct |
| 10 | 10 | ctc tcc acc aac tgc cac at |

Gene fragments were generated by Asymmetric PCR using 10-100 ng of DNA extracted from blood as a template, considering PCR reaction volume to be total 50 μl. The fragments were polymerized by one PCR reaction using different amount of forward and reverse primers in a ratio of 1:5 to 1:10. The first denaturation was carried out at 98° C. for 5 min. just once, and then main 35 cycles of PCR were performed by secondly denaturing at 94° C. for 1 min, annealing at 55° C. for 1 min and extension at 72° C. for 1 min. PCR was terminated by maintaining 72° C. for 7 min once for the last extension.

### Example 3

### Manufacturing Probes for Diagnosis of BIGH3 Gene Mutation

In order to search mutations of corneal dystrophy as shown in Table 1, probes were designed to embrace 5-8 more nucleotide sequences expanding from both sides of a hotspot, more preferably 7 more nucleotides. Probes for normal counterparts were designed to embrace the same sequences as the above but normal instead of mutated nucleotide sequences at the center.

TABLE 3
Probes constructed for diagnosing
mutation of ophthalmologic diseases

| | | | | |
|---|---|---|---|---|
| SEQ | | Normal/ | | |
| ID NO. | Genotype | Mutation | Probe sequence | Exon |

| | | | | |
|---|---|---|---|---|
| 11 | Normal | R124 | acg gac cgc acg gag | 4 |
| 12 | Avellino dystrophy | R124H | acg gac cac acg gag | 4 |
| 13 | Reis-bucklers(CDB1) | R124L | acg gac ctc acg gag | 4 |
| 14 | Normal | R124 | cac gga ccg cac gga | 4 |
| 15 | Lattice type I | R124C | cac gga ctg cac gga | 4 |
| 16 | Normal | P501 | gac ccc ccc aat ggg | 11 |
| 17 | Lattice typelllA | P501T | gac ccc cac aat ggg | 11 |
| 18 | Normal | L518 | agc atg ctg gta gct | 12 |
| 19 | Lattice dystrophy Pro | L518P | agc atg ccg gta gct | 12 |
| 20 | Normal | L527 | gca gga ctg acg gag | 12 |
| 21 | Lattice dystrophy Arg | L527R | gca gga cgg acg gag | 12 |
| 22 | Normal | F540 | aca gtc ttt gct ccc | 12 |
| 23 | Reis-bucklers | F540 | ac aca gtc gct ccc ac | 12 |
| | | deletion | | |
| 24 | Normal | N544 | ccc aca aat gaa gcc | 12 |
| 25 | Lattice dystrophy ser1 | N544S | ccc aca agt gaa gcc | 12 |
| 26 | Normal | N544 | ccc aca aac gaa gcc | 12 |
| 27 | Lattice dystrophy ser2 | N544S | ccc aca agc gaa gcc | 12 |
| 28 | Lattice dystrophy ser3 | N544S | ccc aca tct gaa gcc | 12 |
| 29 | Lattice dystrophy ser4 | N544S | ccc aca tcc gaa gcc | 12 |
| 30 | Lattice dystrophy ser5 | N544S | ccc aca tca gaa gcc | 12 |
| 31 | Lattice dystrophy ser6 | N544S | ccc aca tcg gaa gcc | 12 |
| 32 | Normal | A546 | aaa tga agc cttc cga | 12 |
| 33 | Lattice typelllA thr | A546T | aaa tga aac cttc cga | 12 |
| 34 | Normal | R555 | aag aga acg gag cag | 12 |
| 35 | Granular dystrophy | R555W | aag aga atg gag cag | 12 |
| 36 | Normal | R555 | aga gaa cgg agc aga | 12 |
| 37 | Reis-bucklers(CDB2) | R555Q | aga gaa cag agc aga | 12 |
| 38 | Normal | N622 | ggc cac aaa tgg cgt | 14 |
| 39 | Normal | N622 | ggc cac aaa cgg cgt | 14 |
| 40 | Lattice dystrophy his | N622H | ggc cac aca cgg cgt | 14 |
| 41 | Normal | G623 | aca aat ggc gtg gtc | 14 |
| 42 | Reis-bucklers-1 | G623D | aca aat gat gtg gtc | 14 |
| 43 | Normal | G623 | caa acg gcg tgg tcc | 14 |
| 44 | Reis-bucklers-2 | G623D | caa acg atg tgg tcc | 14 |
| 45 | Normal | H626 | gtg gtc cat gtc atc | 14 |
| 46 | 14Lattice dystrophy | H626R | gtg gtc cgt gtc atc | 14 |

### Example 4

### Manufacturing DNA Chip for Diagnosing Corneal Dystrophy

Each probe selected in Example 3 was synthesized to apply to DNA chip. Mononucleotides (Proligo Biochemie GmbH Hambrug Co. GE) were injected into Automatic synthesizer (Expedite™ 8900, PE Biosystems Co. USA) with information of nucleotide sequences and input scales, and were polymerized to be 0.05 μM of pure nucleic acid probes. The synthesis of resulted probes was confirmed by electrophoresis.

In order to immobilize DNA probes on a solid support, amine-aldehyde covalent bonds were used. The 3′ end of synthetic oligonucleotide probes was modified to insert amine residues using an amino linker column (Cruachem, Glasgow, Scotland) for immobilization, and dissolved in spotting buffer (3×SSC; 0.45 M NaCl; 15 mM C₆H₅Na₃O₇, pH 7.0). The resulting solution was spotted on the aldehyde-coated glass slides (CEL Associates, Inc. Huston, USA) using microarrayer, and then the slides were kept under more than 55% humid condition to allow amine and aldehyde to bind for more than 1 hour, followed by immobilizing DNA probes for 6 hours. Position markers and each probes were fixed at the concentration of 2-5 μM and 10-100 μM, respectively. To evaluate efficiency of immobilization, the glass slides were dyed with SYBRO green II (Molecular Probes, Inc., Leiden, Netherlands).

### Example 5

### Diagnosis of Corneal Dystrophy Using DNA Chip for Diagnosing Thereof

To confirm the specificity and sensitivity of probes, hybridization was performed by applying the PCR products prepared in Example 2 to the DNA chip capturing probes that were generated in Example 4. After hybridizing with genomic DNA extracted from blood of patients or normal individuals, whether a probe produced a positive hybridization signal and crossreactivity (specificity) with other probes were tested.

DNA chip was hydrated with a water vapor and then soaked in 70% ethanol to remove any probes that were not immobilized on the glass of DNA chip. The DNA chip was transferred to a blocking solution (1.3 g of NaBH₄, 375 ml of PBS, 125 ml of 100% ethanol) and shaken for 5 minutes in order to prevent fluorescent chemicals from attaching aldehyde groups on the surface of a glass, which might result in boosting total intensity of fluorescence and cause the eclipse of specific and positive signals. The DNA chip was then washed with 0.2% SDS for 5 min. and ensued by twice or three times with sterile water for 1 min. each. The liquid remains on the glass surface of DNA chip was eliminated by centrifugation (at 1,000 rpm for 2 min).

10-20 μl of asymmetric PCR products was added with hybridization buffer [6×SSPE (0.9M NaCl, 10 mM NaH₂PO₄—H₂O, 1 mM EDTA, pH 7.4) or 20×SSPE (3M NaCl, 0.2M NaH₂PO₄—H₂O, 0.02M EDTA, pH 7.4)], 20% (v/v) formamide (Sigma Co., St. Louis USA) to be total 200 μl. The resulting mixture was applied on a glass slide onto which probes were immobilized and covered with a probe-clip press-seal incubation chamber (Sigma Co., St. Louis, Mo.).

The hybridization reaction was continued for 4-6 hours in shaking incubator at 30° C., preventing dehydration of ambient air around glass by placing wet cotton tissues in the chamber during hybridization to induce complementary binding. After the completion of hybridization, the slides were washed subsequently with 3×SPE (0.45 M NaCl, 15 mM C₆HsNa₃O₇, pH 7.0), 2×SSPE (0.3 M NaCl, 10 mM C₆HsNa₃O₇, pH 7.0) and then 1×SSPE (0.15 M NaCl, 10 mM C₆HsNa₃O₇, pH 7.0) for 5 min. each. The liquid remains on the glass surface of DNA chip was eliminated by centrifugation (at 1,000 rpm for 2 min).

The hybrids were detected by DNA chip scanner (CCD camera based scanner, ArrayWoRx, Applied Precision LLC, USA), with exposure time of 0.2˜0.5 sec. Fluorescence intensity was measured at 595 nm for Cy3, 685 nm for Cy5 and 530 nm for FITC and the detected signals were interpreted by ImaGene 6.0 software (Biodiscovery Inc., USA).

### Example 6

### Detection Efficiency of DNA Chip Depending on Spotting Buffer

The detection efficiency of DNA chip was assessed depending on spotting buffers, in order to manufacture efficient DNA chip that can selectively detect point mutation of one or two nucleotides for diagnosing corneal dystrophy.

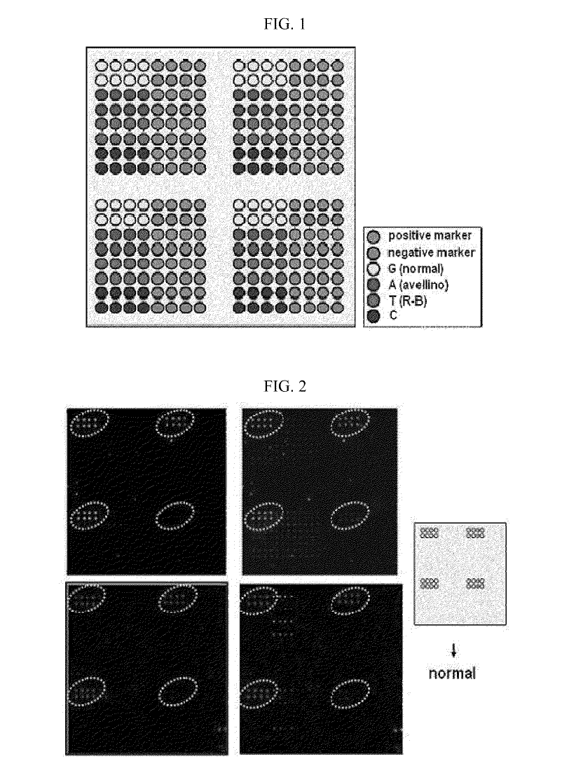

DNA chip was manufactured with different spotting solutions, but in which 0.1 M probes produced in Example 4 are contained. Probes on 2 blocks in the left among 4 blocks as shown in FIG. 1 were spotted with a commercial buffer (Telechem, USA), one block in the upper left was spotted with 3×SSC, and the other block in the lower right, with 50% DMSO solution. Yellow regions represent probes detecting normal individuals, and pink and bluish green represent probes detecting Avellino dystrophy and Reis-bucklers CD1, respectively.

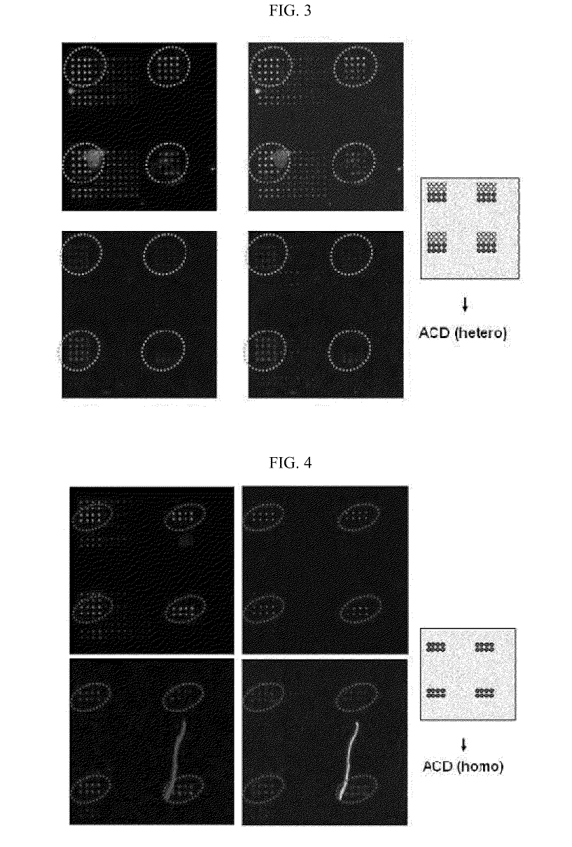

FIG. 2˜FIG. 5 show the result of hybridization using DNA chip designed as described above and exon 4 PCR products. Cy5 (Red) fluorescence stands for the result by applying PCR product that was amplified with primer 1 and primer 2, and Cy3 (green) fluorescence does the result with primer 3 and primer 4. A box located in the right stands for a comparable result, such as FIG. 2 for normal individuals, FIG. 3 for heterozygous Avellino dystrophy, FIG. 4 for homozygous Avellino dystrophy and FIG. 5 for heterozygous Reis-Bucklers CD 1.

As shown in FIG. 2˜FIG. 5, spotting with 3×SSC produced most precise and clear signals, hence, in the following examples, all hybridization was performed with 3×SSC.

### Example 7

### Detection Efficiency of DNA Chip Depending on Probe Concentration and Hybridization Time

The detection efficiency of DNA chip was assessed depending on probe concentration and hybridization time, in order to manufacture efficient DNA chip that can selectively detect point mutation of one or two nucleotides for diagnosing corneal dystrophy.

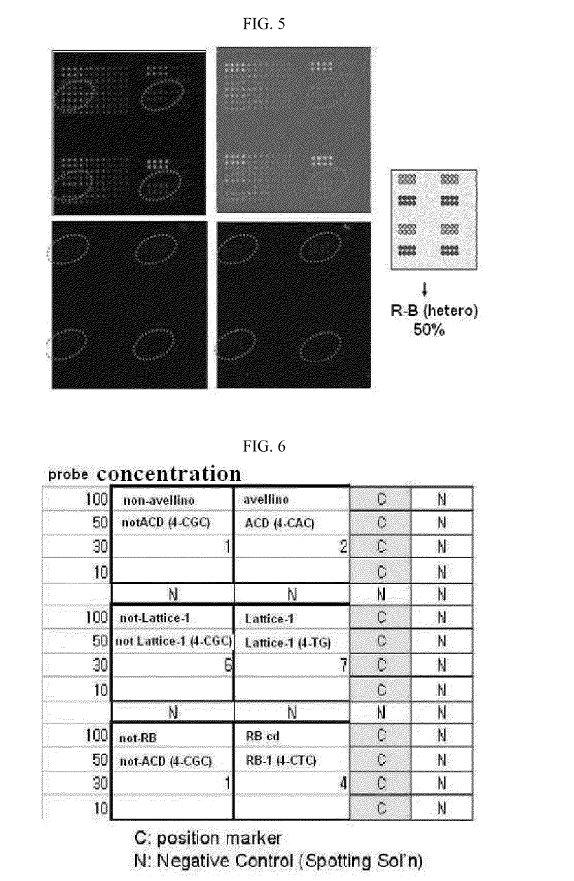

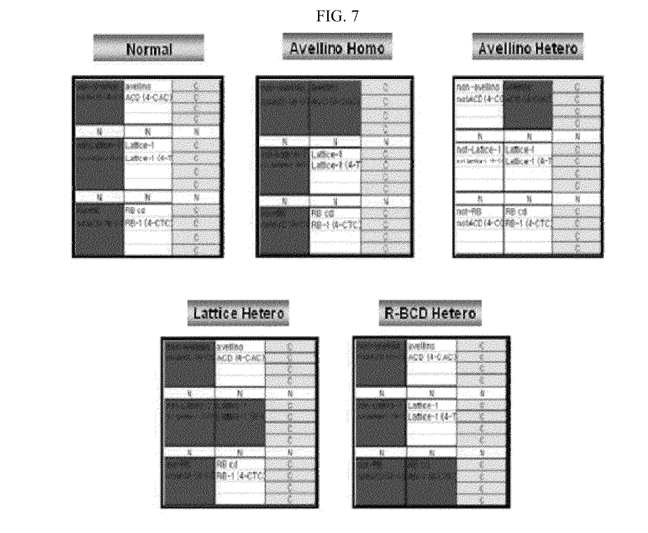

DNA chip was manufactured by spotting 10-100 μM probes produced in Example 4 with 3×SSC. As shown in FIG. 6, one DNA chip was manufactured to contain two blocks. FIG. 7 shows the anticipated result of hybridization depending on patients.

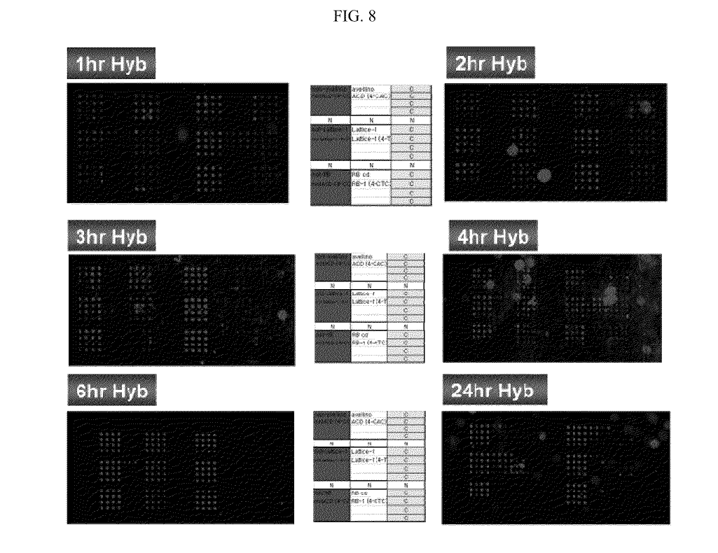

FIG. 8 shows the hybridization result using the DNA chip above and primer sets depending on hybridization time. Red spots are the hybridization result of PCR product amplified with primer 1 and primer 2 and labeled with Cy5. Hybridization time and applied patients were enlisted on each drawing.

As a result of PCR product of exon 4 region amplified with primer 1 and 2, the optimal condition for hybridization turned out that probe concentration was 30-50 μM of probes and hybridization time was recommended for 2-6 hours, more preferably 4-6 hours and most preferably 6 hours.

### Example 8

### Detection Efficiency of DNA Chip Depending on Probe Length

The detection efficiency of DNA chip was assessed depending on probe length, in order to manufacture efficient DNA chip that can selectively detects point mutation of one or two nucleotides for diagnosing Avellino corneal dystrophy, Reis-bucklers I corneal dystrophy, Lattice type I corneal dystrophy and Granular corneal dystrophy.

Based on the mutation sites enumerated in Table 3, variety of probe length such as 11 mer, 12 mer, 15 mer and 17 mer were synthesized, based on the probe sequences of primer 11 to primer 15, and primer 34 and primer 35.

TABLE 4
Probe sequences for determining optimal probe length.

| | | | SEQ | |
|---|---|---|---|---|
| Probe | Length | Sequence | ID NO. | |
| not ACD | 11 | ggaccgcacgg | 47 | Normal/Hetero |
| | 13 | cggaccgcacgga | 48 | |
| | 15 | acggaccgcacggag | 11 | |
| | 17 | cacggaccgcacggaga | 49 | |
| ACD | 11 | ggaccacacgg | 50 | Avellino |
| | 13 | cggaccacacgga | 51 | |
| | 15 | acggaccacacggag | 12 | |
| | 17 | cacggaccacacggaga | 52 | |
| RBCD | 11 | ggacctcacgg | 53 | Reis-Bucklers I |
| | 13 | cggacctcacgga | 54 | |
| | 15 | acggacctcacggag | 13 | |
| | 17 | cacggacctcacggaga | 55 | |
| not LCD I | 11 | cggaccgcacg | 56 | Normal/Hetero |
| | 13 | acggaccgcacgg | 57 | |
| | 15 | cacggaccgcacgga | 14 | |
| | 17 | acacggaccgcacggag | 58 | |
| LCD I | 11 | cggactgcacg | 59 | Lattice type I |
| | 13 | acggactgcacgg | 60 | |
| | 15 | cacggactgcacgga | 15 | |
| | 17 | acacggactgcacggag | 61 | |
| not GCD | 15 | aagagaacggagcag | 34 | Normal/Hetero |
| | 17 | caagagaacggagcaga | 62 | |
| | 16 | caagagaacggagcag | 63 | |
| | 16 | aagagaacggagcaga | 64 | |
| GCD | 17 | caagagaatggagcaga | 65 | Granular type |
| | 16 | caagagaatggagcag | 66 | |
| | 16 | aagagaatggagcaga | 67 | |

DNA chip was manufactured with 3×SSC spotting solution and 50 PM probes produced in Example 4.

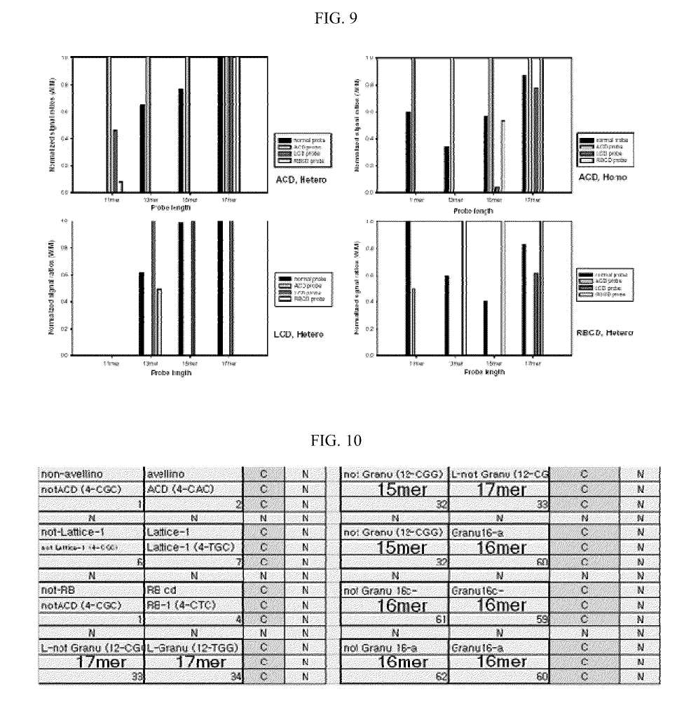

The ratio (W/M) of mutation signals (M) to normal signals (W) was defined by the ratio of spot signals (W) of probes detecting normal sequences to spot signals (M) of probes detecting dystrophy. And the ratio was optimized and interpreted. As shown in FIG. 9, Avellino corneal dystrophy was detected most successfully by 13 mer probe as well as quite efficiently by 15 mer probe. Reis-bucklers I corneal dystrophy and Lattice type I corneal dystrophy were detected most efficiently by 15 mer probe. Therefore, the detection of mutation regions of exon 4 for Avellino corneal dystrophy, Reis-bucklers I corneal dystrophy and Lattice type I corneal dystrophy was conducted with 15 mer probe. In Table 4, the optimal length of probes was emphasized by bold fonts for diagnostic DNA chip of corneal dystrophy.

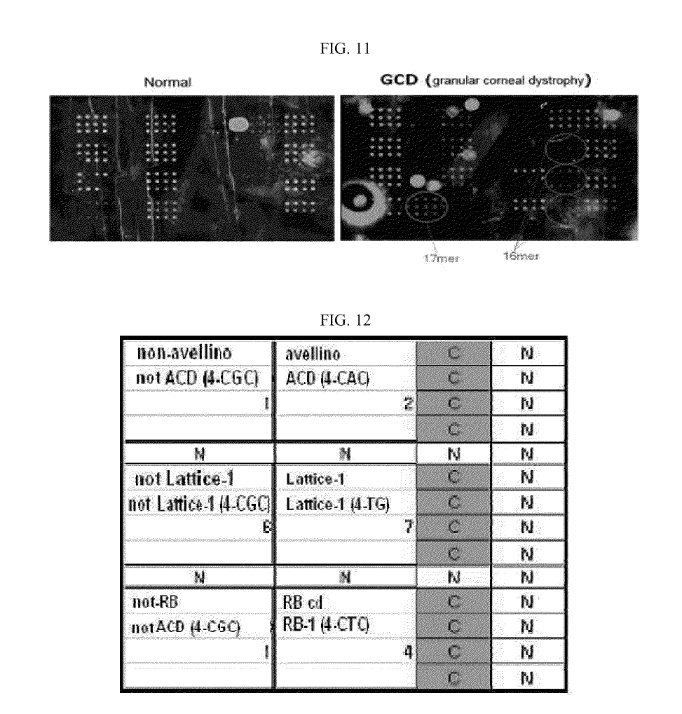

The detection of Granular corneal dystrophy, containing point mutations in exon 12 was performed most effectively with 17 mer probe using DNA chip in FIG. 10 (FIG. 11).

### Example 9

### Hybridization of DNA Chip for Diagnostic Application

Based on the results of Example 6, Example 7 and Example 8, the detection efficiency of DNA chip was assessed depending on probe concentration and hybridization time, in order to manufacture efficient DNA chip that can selectively detect point mutations of one or two nucleotides for diagnosing corneal dystrophy.

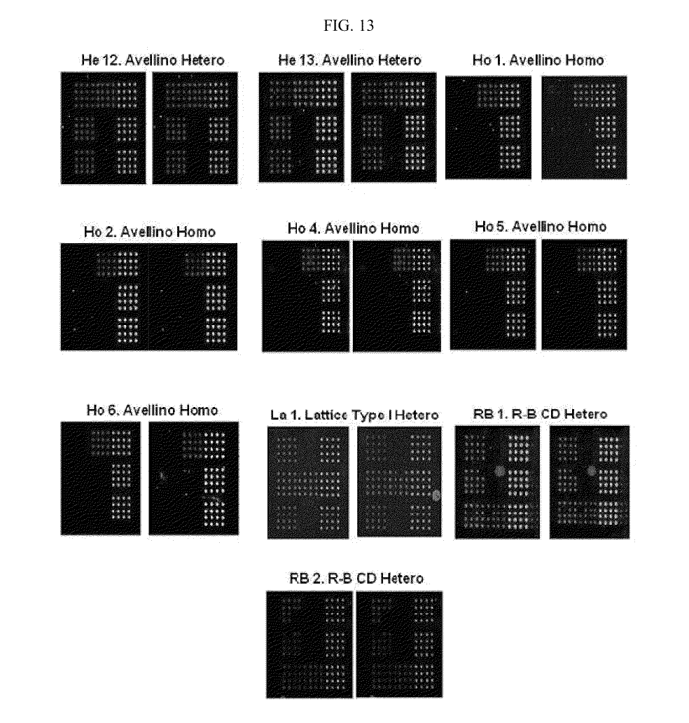

Eventually, DNA chip was manufactured with 3×SSC spotting solution and 50 μM of 15 mer probes (FIG. 12). FIG. 13 shows the anticipated results of hybridization depending on patients.

FIG. 13 shows the result of hybridization using DNA chip manufactured as described above and PCR products of patients, amplified with primer 1 and primer 2. Subjects were enlisted in each drawing. The application result of DNA chip of the present invention to patients in diagnosis confirmed possibility of precise diagnosis for all patients, and proved superiority of specificity as well as sensitivity.

By the result of hybridization, DNA chip was manufactured, which is applicable to diagnosis of ophthalmologic diseases with 15 mer probes detecting for Avellino corneal dystrophy, Reis-bucklers I corneal dystrophy and Lattice type I corneal dystrophy, and 17 mer probes detecting for Granular corneal dystrophy. The procedure, that 30-50 μM probes in 3×SSC was spotted and hybridized at 30° C. for 6 hours, was found to generate optimal diagnostic result. And the result delivered a fact that hybridization can be performed at 15-20° C. higher than Tm corresponding to probe length. Therefore, DNA chip of the present invention can be processed by controlling hybridization temperature that is determined by probe length fixed on the chip, and utilized for diagnosis of corneal dystrophy including Avellino corneal dystrophy caused by BIGH3 mutation.

Using the optimized DNA chip for diagnosis of corneal dystrophy manufactured above, blood samples from 98 patients were subjected to diagnosis of corneal dystrophy. As a result, as shown in Table 5, 27 patients turned out to be normal, 10 patients to be homo Avellino corneal dystrophy, 57 patients to be hetero Avellino corneal dystrophy, 1 patient to be hetero Lattice type I corneal dystrophy, 1 patient to be hetero Reis-Bucklers type I corneal dystrophy, and 2 patients to be Granular corneal dystrophy.

TABLE 5
Diagnosis result of clinical samples

| | | | Mutation | | Pateint. |
|---|---|---|---|---|---|
| Illlness | Mutation | Mutation site | type | Exon | No. |
| Avellino | R124H | CGC→CAC | heterozygous | Exon 4 | 57 |
| Avellino | R124H | CGC→CAC | homozygous | Exon 4 | 10 |
| Lattice type I | R124C | CGC→TGC | heterozygous | Exon 4 | 1 |
| Reis- | R124L | CGC→CTC | heterozygous | Exon 4 | 1 |
| Bucklers I | | | | | |
| Granular | R555W | CGG→TGG | | Exon 12 | 2 |
| type | | | | | |
| normal | None | | normal | | 27 |
| Total | | | | | 98 |

Said diagnosis result was confirmed by DNA sequence analysis of clinical samples, which proved 100% of accuracy.

### INDUSTRIAL APPLICABILITY

As described above in detail, the present invention has an effect of providing oligonucleotides containing a mutation region of BIGH3 gene that causes corneal dystrophy, and a DNA chip for diagnosis of corneal dystrophy, which has the oligonucleotides fixed thereon. According to the present invention, conventional microscopic diagnosis of corneal dystrophy can be replaced with a precise genetic method, which prevents a patient with corneal dystrophy from losing eyesight by eyesight correction surgery after erroneous diagnosis.

Although the present invention has been described in detail with reference to the specific features, it will be apparent to those skilled in the art that this description is solely for a preferred embodiment and does not limit the scope of the present invention. Thus, the substantial scope of the present invention will be defined by the appended claims and equivalents thereof.

1. Oligonucleotides for diagnosis of corneal dystrophy, which essentially comprises one or more nucleotide sequences selected from the group consisting of SEQ ID NO: 17, SEQ ID NO: 19, SEQ ID NO: 21, SEQ ID NO: 23, SEQ ID NO: 25, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 40, SEQ ID NO: 42, SEQ ID NO:44, SEQ ID NO:46, SEQ ID NO: 50, SEQ ID NO:53 and SEQ ID NO: 59.

2. The oligonucleotide for diagnosis of corneal dystrophy according to claim 1, wherein said oligonucleotide essentially comprising the nucleotide sequence of SEQ ID NO: 50 is set forth in SEQ ID NO: 12.

3. The oligonucleotide for diagnosis of corneal dystrophy according to claim 1, wherein said oligonucleotide essentially comprising the nucleotide sequence of SEQ ID NO: 53 is set forth in SEQ ID NO: 13.

4. The oligonucleotide for diagnosis of corneal dystrophy according to claim 1, wherein said oligonucleotide essentially comprising the nucleotide sequence of SEQ ID NO: 59 is set forth in SEQ ID NO: 15.

5. The oligonucleotide for diagnosis of corneal dystrophy according to claim 1, wherein said oligonucleotide essentially comprising the nucleotide sequence of SEQ ID NO: 35 is set forth in SEQ ID NO: 65.

6. The oligonucleotide for diagnosis of corneal dystrophy according to claim 1, wherein the length of the oligonucleotide is 13 to 17 bp.

7. A DNA chip for diagnosis of corneal dystrophy, wherein the oligonucleotide of claim 1 is fixed on the substrate thereof.

8. The DNA chip for diagnosis of corneal dystrophy according to claim 7, which additionally immobilizes oligonucleotides, essentially comprising one or more nucleotide sequences selected from the group consisting of SEQ ID NO: 16, SEQ ID NO: 18, SEQ ID NO: 20, SEQ ID NO: 22, SEQ ID NO: 24, SEQ ID NO: 26, SEQ ID NO: 32, SEQ ID NO: 34, SEQ ID NO: 36, SEQ ID NO: 38, SEQ ID NO: 39, SEQ ID NO: 41, SEQ ID NO: 43, SEQ ID NO: 45, SEQ ID NO: 47, and SEQ ID NO:56, thereon.

9. The DNA chip for diagnosis of corneal dystrophy according to claim 8, wherein said oligonucleotide essentially comprising the nucleotide sequence of SEQ ID NO: 47 is set forth in SEQ ID NO: 11.

10. The DNA chip for diagnosis of corneal dystrophy according to claim 8, wherein said oligonucleotide essentially comprising the nucleotide sequence of SEQ ID NO: 56 is set forth in SEQ ID NO: 14.

11. The DNA chip for diagnosis of corneal dystrophy according to claim 8, wherein said oligonucleotide essentially comprising the nucleotide sequence of SEQ ID NO: 34 is set forth in SEQ ID NO: 62.

12. The DNA chip for diagnosis of corneal dystrophy according to claim 7, which immobilizes all oligonucleotides, essentially comprising the nucleotide sequence of the group consisting of SEQ ID NO: 16 to SEQ ID NO: 47, SEQ ID NO: 50, SEQ ID NO: 53, SEQ ID NO: 56 and SEQ ID NO:59, thereon.

13. A DNA chip for diagnosis of corneal dystrophy, wherein all oligonucleotides of the group consisting of SEQ ID NO: 11 to SEQ ID NO:15, SEQ ID NO: 62 and SEQ ID NO: 65 are fixed thereon.

14. A pair of primers selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; and SEQ ID NO: 9 and SEQ ID NO: 10.


SEQUENCE LISTING (67 sequences)

SEQ ID NO: 1 - DNA, 20 bp, Artificial Sequence
  Primer
agcagccctaccactctcaa

SEQ ID NO: 2 - DNA, 18 bp, Artificial Sequence
  Primer
caggcctcgttgctaggg

SEQ ID NO: 3 - DNA, 20 bp, Artificial Sequence
  Primer
ccccagaggccatccctcct

SEQ ID NO: 4 - DNA, 20 bp, Artificial Sequence
  Primer
ccgggcagacggaggtcatc

SEQ ID NO: 5 - DNA, 20 bp, Artificial Sequence
  Primer
ctcgtgggagtataaccagt

SEQ ID NO: 6 - DNA, 20 bp, Artificial Sequence
  Primer
tgggcagaagctccacccgg

SEQ ID NO: 7 - DNA, 27 bp, Artificial Sequence
  Primer
cattccagtggcctggactctactatc

SEQ ID NO: 8 - DNA, 23 bp, Artificial Sequence
  Primer
ggggccctgagggatcactactt

SEQ ID NO: 9 - DNA, 20 bp, Artificial Sequence
  Primer
ctgttcagtaaacacttgct

SEQ ID NO: 10 - DNA, 20 bp, Artificial Sequence
  Primer
ctctccaccaactgccacat

SEQ ID NO: 11 - DNA, 15 bp, Artificial Sequence
  Probe
acggaccgcacggag

SEQ ID NO: 12 - DNA, 15 bp, Artificial Sequence
  Probe
acggaccacacggag

SEQ ID NO: 13 - DNA, 15 bp, Artificial Sequence
  Probe
acggacctcacggag

SEQ ID NO: 14 - DNA, 15 bp, Artificial Sequence
  Probe
cacggaccgcacgga

SEQ ID NO: 15 - DNA, 15 bp, Artificial Sequence
  Probe
cacggactgcacgga

SEQ ID NO: 16 - DNA, 15 bp, Artificial Sequence
  Probe
gacccccccaatggg

SEQ ID NO: 17 - DNA, 15 bp, Artificial Sequence
  Probe
gacccccacaatggg

SEQ ID NO: 18 - DNA, 15 bp, Artificial Sequence
  Probe
agcatgctggtagct

SEQ ID NO: 19 - DNA, 15 bp, Artificial Sequence
  Probe
agcatgccggtagct

SEQ ID NO: 20 - DNA, 15 bp, Artificial Sequence
  Probe
gcaggactgacggag

SEQ ID NO: 21 - DNA, 15 bp, Artificial Sequence
  Probe
gcaggacggacggag

SEQ ID NO: 22 - DNA, 15 bp, Artificial Sequence
  Probe
acagtctttgctccc

SEQ ID NO: 23 - DNA, 16 bp, Artificial Sequence
  Probe
acacagtcgctcccac

SEQ ID NO: 24 - DNA, 15 bp, Artificial Sequence
  Probe
cccacaaatgaagcc

SEQ ID NO: 25 - DNA, 15 bp, Artificial Sequence
  Probe
cccacaagtgaagcc

SEQ ID NO: 26 - DNA, 15 bp, Artificial Sequence
  Probe
cccacaaacgaagcc

SEQ ID NO: 27 - DNA, 15 bp, Artificial Sequence
  Probe
cccacaagcgaagcc

SEQ ID NO: 28 - DNA, 15 bp, Artificial Sequence
  Probe
cccacatctgaagcc

SEQ ID NO: 29 - DNA, 15 bp, Artificial Sequence
  Probe
cccacatccgaagcc

SEQ ID NO: 30 - DNA, 15 bp, Artificial Sequence
  Probe
cccacatcagaagcc

SEQ ID NO: 31 - DNA, 15 bp, Artificial Sequence
  Probe
cccacatcggaagcc

SEQ ID NO: 32 - DNA, 16 bp, Artificial Sequence
  Probe
aaatgaagccttccga

SEQ ID NO: 33 - DNA, 16 bp, Artificial Sequence
  Probe
aaatgaaaccttccga

SEQ ID NO: 34 - DNA, 15 bp, Artificial Sequence
  Probe
aagagaacggagcag

SEQ ID NO: 35 - DNA, 15 bp, Artificial Sequence
  Probe
aagagaatggagcag

SEQ ID NO: 36 - DNA, 15 bp, Artificial Sequence
  Probe
agagaacggagcaga

SEQ ID NO: 37 - DNA, 15 bp, Artificial Sequence
  Probe
agagaacagagcaga

SEQ ID NO: 38 - DNA, 15 bp, Artificial Sequence
  Probe
ggccacaaatggcgt

SEQ ID NO: 39 - DNA, 15 bp, Artificial Sequence
  Probe
ggccacaaacggcgt

SEQ ID NO: 40 - DNA, 15 bp, Artificial Sequence
  Probe
ggccacacacggcgt

SEQ ID NO: 41 - DNA, 15 bp, Artificial Sequence
  Probe
acaaatggcgtggtc

SEQ ID NO: 42 - DNA, 15 bp, Artificial Sequence
  Probe
acaaatgatgtggtc

SEQ ID NO: 43 - DNA, 15 bp, Artificial Sequence
  Probe
caaacggcgtggtcc

SEQ ID NO: 44 - DNA, 15 bp, Artificial Sequence
  Probe
caaacgatgtggtcc

SEQ ID NO: 45 - DNA, 15 bp, Artificial Sequence
  Probe
gtggtccatgtcatc

SEQ ID NO: 46 - DNA, 15 bp, Artificial Sequence
  Probe
gtggtccgtgtcatc

SEQ ID NO: 47 - DNA, 11 bp, Artificial Sequence
  Probe
ggaccgcacgg

SEQ ID NO: 48 - DNA, 13 bp, Artificial Sequence
  Probe
cggaccgcacgga

SEQ ID NO: 49 - DNA, 17 bp, Artificial Sequence
  Probe
cacggaccgcacggaga

SEQ ID NO: 50 - DNA, 11 bp, Artificial Sequence
  Probe
ggaccacacgg

SEQ ID NO: 51 - DNA, 13 bp, Artificial Sequence
  Probe
cggaccacacgga

SEQ ID NO: 52 - DNA, 17 bp, Artificial Sequence
  Probe
cacggaccacacggaga

SEQ ID NO: 53 - DNA, 11 bp, Artificial Sequence
  Probe
ggacctcacgg

SEQ ID NO: 54 - DNA, 13 bp, Artificial Sequence
  Probe
cggacctcacgga

SEQ ID NO: 55 - DNA, 17 bp, Artificial Sequence
  Probe
cacggacctcacggaga

SEQ ID NO: 56 - DNA, 11 bp, Artificial Sequence
  Probe
cggaccgcacg

SEQ ID NO: 57 - DNA, 13 bp, Artificial Sequence
  Probe
acggaccgcacgg

SEQ ID NO: 58 - DNA, 17 bp, Artificial Sequence
  Probe
acacggaccgcacggag

SEQ ID NO: 59 - DNA, 11 bp, Artificial Sequence
  Probe
cggactgcacg

SEQ ID NO: 60 - DNA, 13 bp, Artificial Sequence
  Probe
acggactgcacgg

SEQ ID NO: 61 - DNA, 17 bp, Artificial Sequence
  Probe
acacggactgcacggag

SEQ ID NO: 62 - DNA, 17 bp, Artificial Sequence
  Probe
caagagaacggagcaga

SEQ ID NO: 63 - DNA, 16 bp, Artificial Sequence
  Probe
caagagaacggagcag

SEQ ID NO: 64 - DNA, 16 bp, Artificial Sequence
  Probe
aagagaacggagcaga

SEQ ID NO: 65 - DNA, 17 bp, Artificial Sequence
  Probe
caagagaatggagcaga

SEQ ID NO: 66 - DNA, 16 bp, Artificial Sequence
  Probe
caagagaatggagcag

SEQ ID NO: 67 - DNA, 16 bp, Artificial Sequence
  Probe
aagagaatggagcaga